(19) United States
(12) Patent Application Publication
(10) Pub. No.: US 2012/0014898 A1
(43) Pub. Date: Jan. 19, 2012

(54) COMPOSITION FOR PREVENTING HAIR LOSS AND STIMULATING HAIR GROWTH

(76) Inventor: Gee-Hwoon LEE, Seoul (KR)

(21) Appl. No.: 12/846,312

(22) Filed: Jul. 29, 2010

(30) Foreign Application Priority Data
Jul. 13, 2010 (KR) 10-2010-0067621

(51) Int. Cl. A61K 8/49; A61Q 7/00; A61Q 5/00; C07D 309/32

(52) U.S. Cl. 424/70.1; 549/419; 514/459

(57) ABSTRACT

The present invention relates to the use of a composition comprising a compound of the formula I:

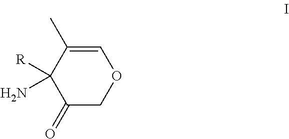

wherein R is C₂H₅ or C₂H₃,
or a pharmaceutically acceptable salt thereof, for preventing hair loss or stimulating hair growth.

### CROSS-REFERENCE TO RELATED APPLICATIONS

This application claims the benefit and priority of Korean Application No. 10-2010-0067621, filed Jul. 13, 2010. The entire disclosure of the above application is incorporated herein by reference.

### TECHNICAL FIELD

The present invention relates to the use of a composition comprising a compound of the formula I or a pharmaceutically acceptable salt thereof, for preventing loss of hair or stimulating hair growth.

### BACKGROUND ART

Baldness is the state of having no hair or lacking hair where it often grows, especially on the head. When one has faulty hair follicles with no possibility of hair growing out of them and his/her frontal hair line is regressed, he/she is in the state of baldness. Baldness can be divided into two categories—Alopecia greata (circular balding) which is caused by the malfunction of endocrine system and androgenetic alopecia, expression of genetic disposition.

The most common form of baldness is a progressive hair thinning condition called androgenic alopecia or “male pattern baldness” that occurs in adult male humans and other species. There are two drugs, Finasteride and Minoxidil, that have been approved by the FDA. However, these drugs can merely retard the progression of hair loss, but do not solve the root cause of hair loss. In addition, hair loss may cause the person to limit social activities, and surveys have shown that around 40% of women with alopecia have had marital problems. In extreme circumstances, some people really take hair loss badly and get highly distressed about it, up to the point of getting into depression. Furthermore, a hair transplant surgery is too expensive so that it is hard for the public to undergo it.

### DISCLOSURE

### Technical Problem

Thus, there still remains a need in the art to fundamentally treat or prevent hair loss as well as retard the progression of hair loss.

The inventor has conducted experiments using nude mice (nu/nu) characterized by hairless appearance due to spontaneous mutation, and discovered that the composition comprising a compound of the formula I prevents loss of hair and stimulates hair growth.

### Technical Solution

The present invention relates to the use of a composition comprising a compound of the formula I:

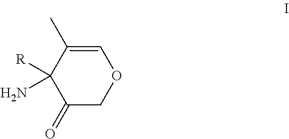

wherein R is C₂H₅ or C₂H₃,
or a pharmaceutically acceptable salt thereof, for preventing loss of hair or stimulating hair growth.

The dose and duration of the treatment will depend on a variety of factors, including the age, body weight, general health, sex, diet, the type of hair loss of the patient. Preferably, the composition according to the invention can comprise an amount of 0.3 mg or more of compound of the formula I relative to 1 ml of the composition. Preferably, the composition according to the invention can be administered at single-dose level of 50 ml to 500 ml, and one time to twelve times a day. When administered twelve times a day, it is desirable to be administered every 2 hours.

The composition according to the invention can be administered by all types of route available in the art. For example, the composition according to the invention can be administered by parenteral (e.g. subcutaneously, intramuscularly, intravenously, intraperitoneally, intrapleurally, intravesicularly or intrathecally), topical, oral, rectal, nasal route, etc.

For the topical application, the composition according to the invention can be in the form of an aqueous, aqueous-alcoholic or oily solution or of a dispersion of the lotion or serum type, of emulsions having a liquid or semi-liquid consistency of the milk type, obtained by dispersion of oil in water, or water in oil, or of suspensions or emulsions of the aqueous or anhydrous cream or gel type, or else of microcapsules or microparticles, or of vesicular dispersions of ionic or non-ionic type. It can also be in the form of an ointment, dye, cream, pomade, powder, patch, impregnated pad, solution, emulsion or vesicular dispersion, lotion, gel, spray, suspension, shampoo, aerosol or foam. It can also consist of solid preparations that form soaps or cleansing bars. And, the composition according to the invention can be formulated in shampoo, lotion, cream, gel, conditioner, essence, etc.

In addition, the composition according to the invention can contain adjuvants that are conventional in the art, such as hydrophilic or lipophilic gelling agents, hydrophilic or lipophilic additives, preservatives, antioxidants, solvents, fragrances, fillers, screening agents, odor absorbers and coloring materials.

The composition according to the invention applies to the skin, preferably the scalp.

### Advantageous Effects

The composition comprising a compound of the formula I according to the invention can treat or prevent loss of hair, and stimulate hair growth. Furthermore, it can even enable hair to grow in bald patients, and thus replace an expensive hair transplant surgery.

### DESCRIPTION OF DRAWINGS

FIG. 1 shows the ¹H NMR spectrum of the compound of the formula II.

FIG. 2 shows the ¹H NMR spectrum of the compound of the formula III.

FIG. 3 is the photograph taken at 0 days after the composition according to the invention was administered to 5-week-old female nude mouse (BALB/c nu/nu).

FIG. 4 is the photograph taken at 7 days after the composition according to the invention was administered to a 5-week-old female nude mouse (BALB/c nu/nu).

FIG. 5 is the photograph taken at 9 days after the composition according to the invention was administered to a 5-week-old female nude mouse (BALB/c nu/nu).

FIG. 6 is the photograph taken at 11 days after the composition according to the invention was administered to a 5-week-old female nude mouse (BALB/c nu/nu).

FIG. 7 is the photograph taken at 13 days after the composition according to the invention was administered to a 5-week-old female nude mouse (BALB/c nu/nu).

FIG. 8 is the photograph taken at 15 days after the composition according to the invention was administered to a 5-week-old female nude mouse (BALB/c nu/nu).

FIG. 9 is the photograph taken at 9 days after the composition according to the invention was administered to another 5-week-old female nude mouse (BALB/c nu/nu).

FIG. 10 is the photograph taken at 15 days after the composition according to the invention was administered to another 5-week-old female nude mouse (BALB/c nu/nu).

FIG. 11 is the graph showing body weight changes in male and female Sprague-Dawley rats after the composition according to the invention is administered.

### EXAMPLES

The present invention is described in further detail in the following Examples which are not in any way intended to limit the scope of the invention as claimed. In addition, it will appear to those ordinarily skilled in the art that various modifications may be made to the disclosed embodiments, and that such modifications are intended to be within the scope of the present invention.

### Example 1

2-methyl-butyric acid and alanine in a weight ratio of 2:1 were reacted at 120° C. and 3.0 atm for 30 minutes. After that, copper and chlorogenic acid in a weight ratio of 3:1 were added to the products obtained by said reaction, and were then reacted at 120˜170° C. and 2.6˜3.0 atm for 10 minutes. After that, hesperidin and water in a weight ratio of 1:1 were added to the products obtained by said reaction, and were then reacted at 80˜120° C. and 2.7-3.5 atm for 8 minutes. After that, sinigrin was added to the products obtained by said reaction, and was then reacted at 100° C. and 2.0 atm for 10 minutes. After that, valine was added to the products obtained by said reaction, and was then reacted at −10˜30° C. and 2˜7 atm for 10 minutes, and in succession was reacted at 200˜230° C. and 1 atm for 10 minutes. In all of the reaction steps, water was used as the solvent.

The final resultant products were purified by two different processes respectively.

As the first purifying process, the final resultant products were dissolved in water at 120° C., and then filtered using the 25 mm nylon syringe filter with 0.2 μm pore size, purchased from VWR. Then, a filtration was successionally performed using the same filter at the temperature of 80° C., 60° C., 30° C. and 12° C., respectively. After that, the filtered solution was vacuum-dried to obtain solid compounds.

As another purifying process, the final resultant products were dissolved in water at 120° C., and then filtered using the 25 mm nylon syringe filter with 0.2 μm pore size, purchased from VWR. Then, a filtration was successionally performed using the same filter at the temperature of 80° C. and 25° C., respectively. After that, the filtered solution was vacuum-dried to obtain solid compounds.

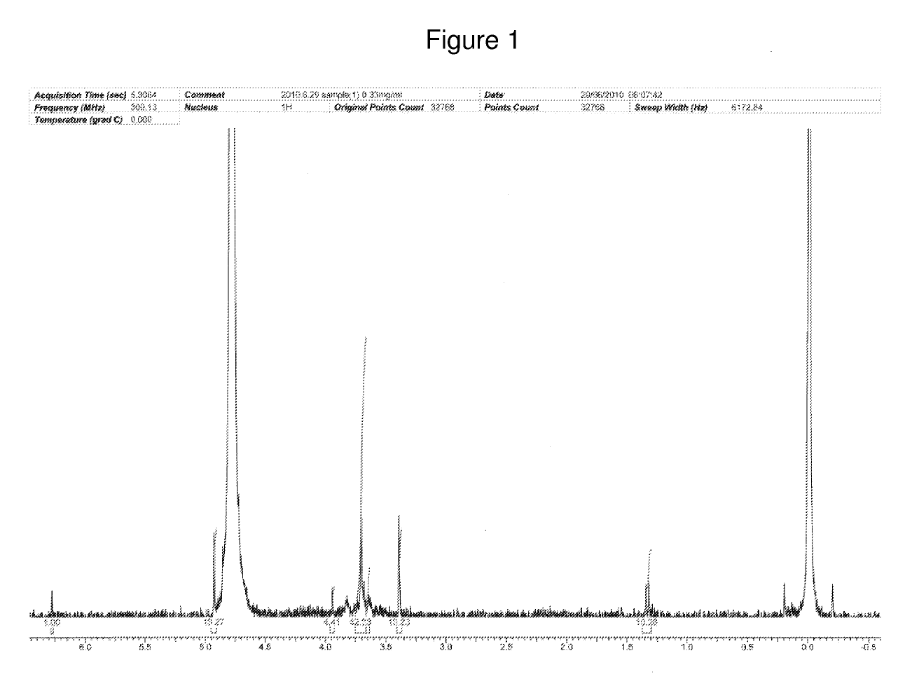

Both compounds obtained by said two processes show the ¹H NMR spectrum of FIG. 1, and are determined to have the following formula II:

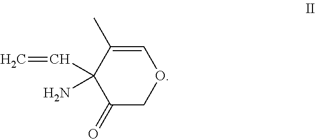

### Example 2

2-methyl-butyric acid and alanine in a weight ratio of 2:1 were reacted at 80˜120° C. and 2.7˜3.0 atm for 10 minutes. After that, copper and chlorogenic acid in a weight ratio of 3:1 were added to the products obtained by said reaction, and were then reacted at 120˜170° C. and 2.6˜3.0 atm for 10 minutes. After that, hesperidin and water in a weight ratio of 1:1 were added to the products obtained by said reaction, and were then reacted at 80˜120° C. and 2.7˜3.5 atm for 8 minutes. After that, sinigrin was added to the products obtained by said reaction, and was then reacted at 120˜140° C. and 3.0˜5.0 atm for 5 minutes. After that, valine was added to the products obtained by said reaction, and was then reacted at −10˜30° C. and 2˜7 atm for 10 minutes, and in succession was reacted at 80˜130° C. and 2˜7 atm for 5 minutes. In all of the reaction steps, water was used as the solvent.

The final resultant products were purified by the two kinds of processes respectively, same as in the Example 1.

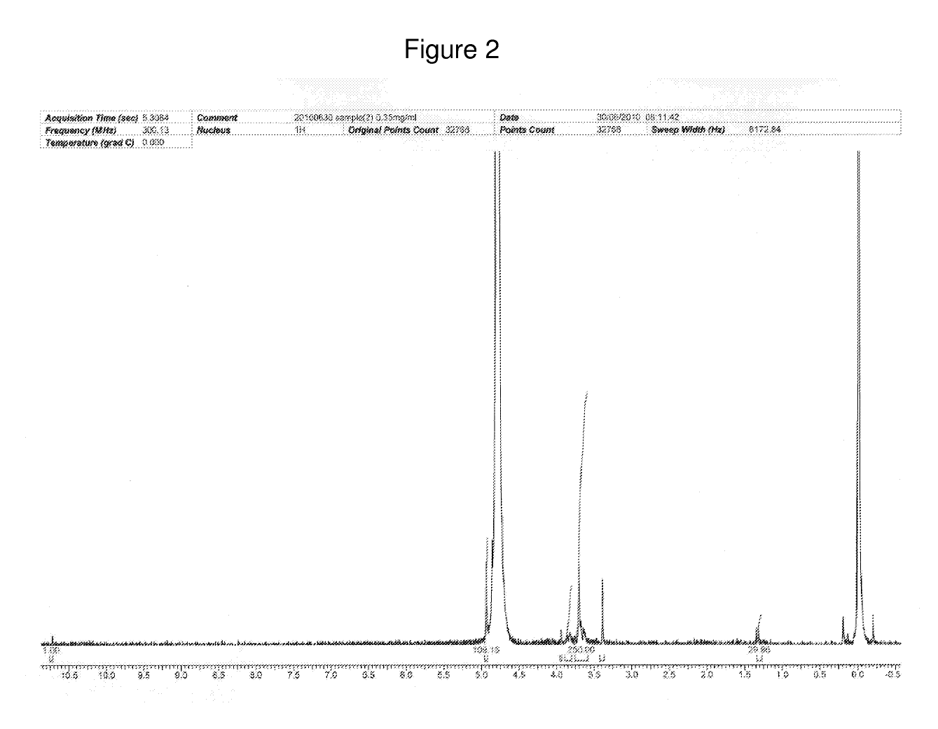

Both compounds obtained by said two processes show the ¹H NMR spectrum of FIG. 2, and are determined to have the following formula III:

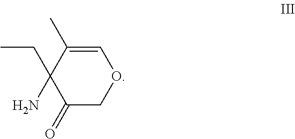

### Example 3

2-methyl-butyric acid and alanine in a weight ratio of 2:1 were reacted at 80˜120° C. and 2.7˜3.0 atm for 10 minutes. After that, copper and chlorogenic acid in a weight ratio of 3:1 were added to the products obtained by said reaction, and were then reacted at 120˜170° C. and 2.6˜3.0 atm for 10 minutes. After that, hesperidin and kaempferol in a weight ratio of 1:1 were added to the products obtained by said reaction, and were then reacted at 80˜120° C. and 1.3 atm for 8 minutes. After that, sinigrin was added to the products obtained by said reaction, and was then reacted at 120˜140° C. and 3.0˜5.0 atm for 5 minutes. After that, valine was added to the products obtained by said reaction, and was then reacted at −10˜30° C. and 2˜7 atm for 10 minutes, and in succession was reacted at 80˜130° C. and 2˜7 atm for 5 minutes. In all of the reaction steps, water was used as the solvent.

The final resultant products were purified by the two kinds of processes respectively, same as in the Example 1. Both compounds obtained by said two processes show the ¹H NMR spectrum of FIG. 2, and are determined to have the formula III.

### Example 4

2-methyl-butyric acid and alanine in a weight ratio of 2:1 were reacted at 80˜120° C. and 2.7˜3.0 atm for 10 minutes. After that, copper and chlorogenic acid in a weight ratio of 3:1 were added to the products obtained by said reaction, and were then reacted at 120˜170° C. and 2.6˜3.0 atm for 10 minutes. After that, hesperidin and 3′-hydroxyformononetin in a weight ratio of 1:1 were added to the products obtained by said reaction, and were then reacted at 120° C. and 2.7 atm for 5 minutes. After that, sinigrin was added to the products obtained by said reaction, and was then reacted at 120˜140° C. and 3.0˜5.0 atm for 5 minutes. After that, valine was added to the products obtained by said reaction, and was then reacted at −10˜30° C. and 2˜7 atm for 10 minutes, and in succession was reacted at 80˜130° C. and 2˜7 atm for 5 minutes. In all of the reaction steps, water was used as the solvent.

The final resultant products were purified by the two kinds of processes respectively, same as in the Example 1. Both compounds obtained by said two processes show the ¹H NMR spectrum of FIG. 2, and are determined to have the formula III.

### Example 5

2-methyl-butyric acid and arctigenin-4-O-glucoside in a weight ratio of 2:1 were reacted at 80˜120° C. and 2.7˜3.0 atm for 10 minutes. After that, copper and luteolin-7-rhamnoglucoside in a weight ratio of 3:1 were added to the products obtained by said reaction, and were then reacted at 120˜170° C. and 2.7 atm for 10 minutes. After that, vitexicarpin and 3′-hydroxyformononetin in a weight ratio of 1:1 were added to the products obtained by said reaction, and were then reacted at 120° C. and 2.7 atm for 5 minutes. After that, sinigrin was added to the products obtained by said reaction, and was then reacted at 120˜140° C. and 3.0-5.0 atm for 5 minutes. After that, valine was added to the products obtained by said reaction, and was then reacted at −10˜30° C. and 2˜7 atm for 10 minutes, and in succession was reacted at 80˜130° C. and 2˜7 atm for 5 minutes. In all of the reaction steps, water was used as the solvent.

The final resultant products were purified by the two kinds of processes respectively, same as in the Example 1. Both compounds obtained by said two processes show the ¹H NMR spectrum of FIG. 2, and are determined to have the formula III.

### Example 6

### Hairs in Nude Mice

The compound of formula II obtained in the Example 1 and the compound of formula III obtained in the Examples 2 to 5 were mixed with purified water, respectively. The prepared liquid-phase composition was at the concentration of 0.33 mg of compound per 1 ml of solution. In the following, the value for the composition according to the invention indicates the mean value of values obtained using the composition comprising the compound of formula II and of formula III.

It is generally known in the art that the two main defects of mice homozygous for the nude spontaneous mutation (Foxnl^{nu}) are abnormal hair growth and defective development of the thymic epithelium. Although the mice appear hairless, they are born with functional but faulty hair growth follicles. Hair growth cycles and patterns are evident especially in pigmented mice but the faulty follicles do not allow the hair to properly erupt.

During the experiment, the composition according to the invention was orally administered to 5-week-old female nude mice (BALB/c nu/nu) at dose level of 0.00033 mg/g (weight of compound/body weight of mouse) twice a day for 2 weeks. The group was consisted of 10 nude mice.

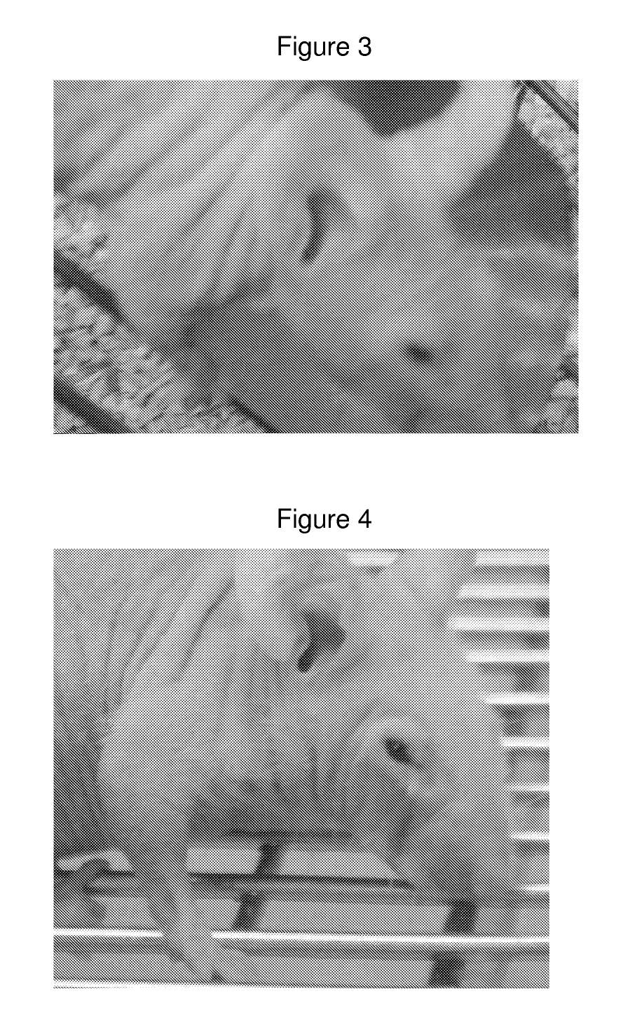

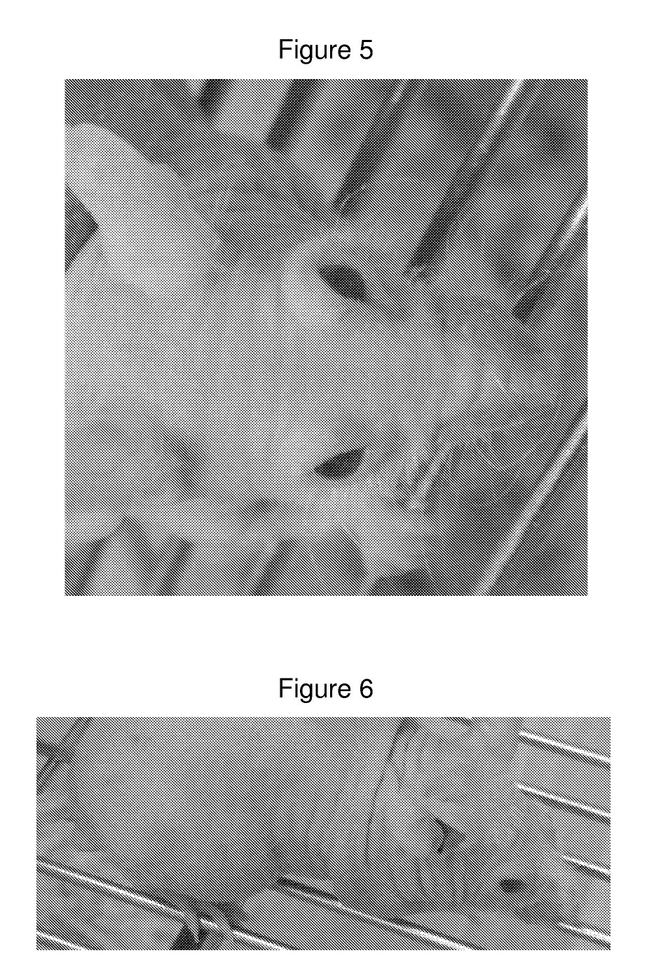

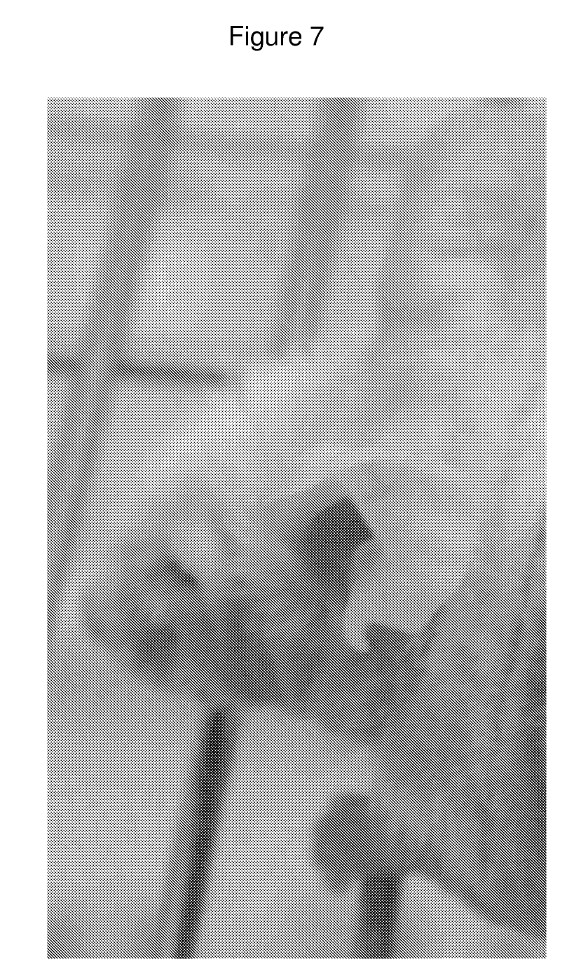

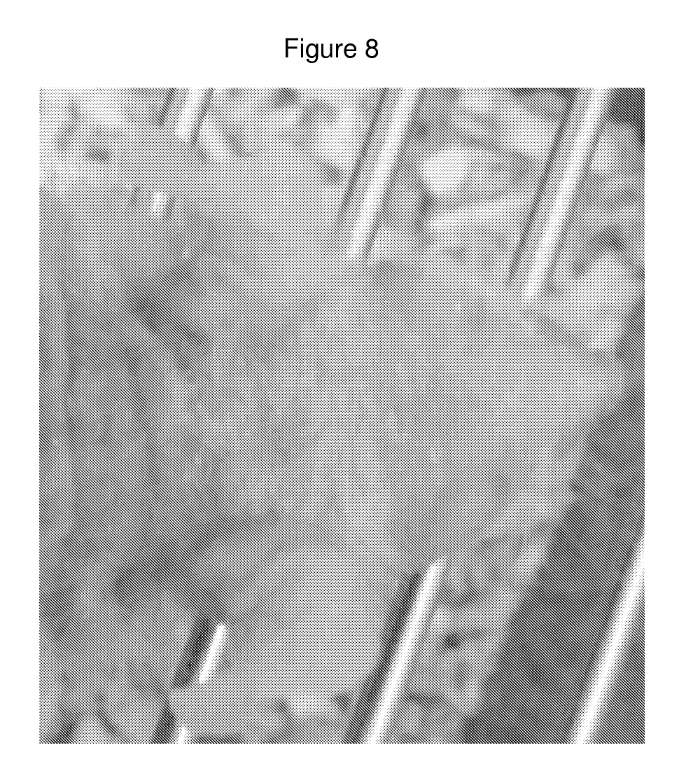

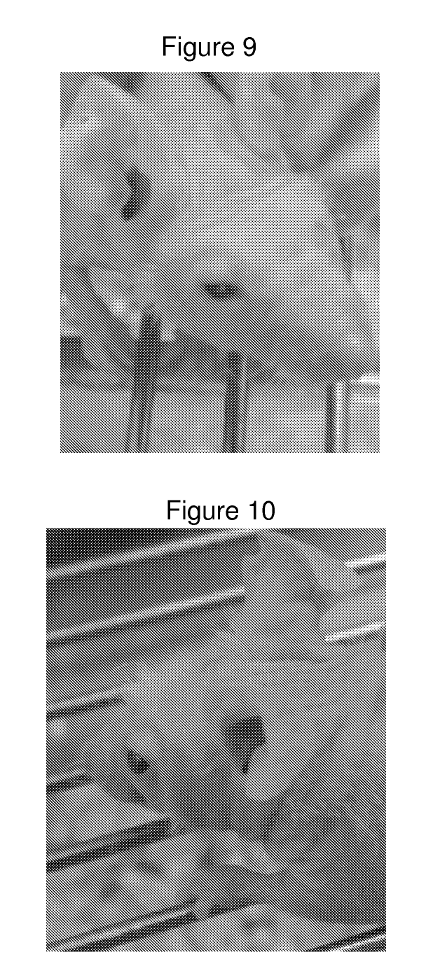

As a result, it is surprisingly found in all of the hairless nude mice that their hair started to grow on heads and flanks, as time goes by. In particular, at two weeks after administration of the composition according to the invention, it was observed that hair grew healthier and fuller in the nude mice. FIGS. 3 to 8 show the photographs taken at 0, 7, 9, 11, 13 and 15 days after administered to no. 1 nude mouse, respectively. FIG. 9 shows the photograph taken at 9 days after administration to no. 2 nude mouse, and FIG. 10 shows the photograph taken at 15 days after administration to no. 7 nude mouse. This phenomenon means that abnormal genes in nude mice, after being treated with the composition according to the invention, changed into normal ones in wild type mice having white hair.

### Example 7

### In Vivo Toxicity Test in Rats

The present experiment was carried out to evaluate the single-dose oral toxicity of the composition according to the invention, in Sprague-Dawley rats.

The composition according to the invention was administered to male and female rats at dose level of 80 ml/kg (volume of composition/body weight of rat), i.e. 28 mg/Kg (weight of compound/body weight of rat). Vehicle control groups treated with distilled water were set up. Each group was consisted of 5 rats of each sex. Mortalities, clinical signs and body weight changes were monitored for 14 days. At the end of 14-day observation period, all animals were sacrificed and necropsy findings were observed. The results are as follows:

(1) No dead animals were observed during the experimental period.

(2) No abnormal clinical signs were observed.

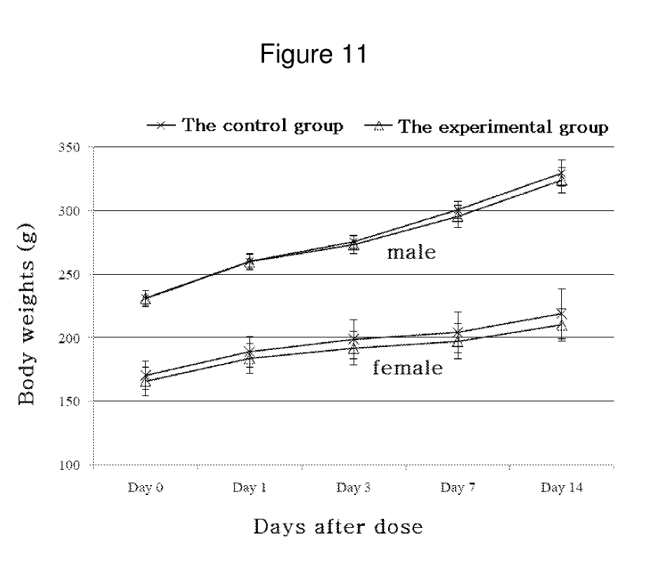

(3) There were no notable test article-related changes in body weight (FIG. 11).

(4) No test article-related abnormal gross findings were observed.

What is claimed is:

1. A composition for preventing hair loss or stimulating hair growth, comprising a compound of the formula I: 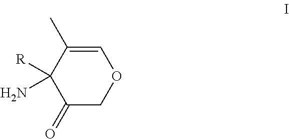
wherein R is C₂H₅ or C₂H₃,
or a pharmaceutically acceptable salt thereof.

2. The composition according to claim 1, wherein the composition comprises an amount of 0.3 mg or more of compound of the formula I relative to 1 ml of the composition.

3. The composition according to claim 1, wherein the composition is administered at single-dose level of 50 ml to 500 ml, and one time to twelve times a day.

4. The composition according to claim 1, wherein the composition is administered by parenteral, topical, oral, rectal or nasal route.

5. The composition according to claim 1, wherein the composition is formulated in shampoo, lotion, cream, gel, conditioner or essence.

6. The composition according to claim 1, wherein the composition contains hydrophilic or lipophilic gelling agents, hydrophilic or lipophilic additives, preservatives, antioxidants, solvents, fragrances, fillers, screening agents, odor absorbers or coloring materials.

7. The composition according to claim 1, wherein the composition applies to the scalp.

8. A method for preventing hair loss or stimulating hair growth in a human or animal subject, comprising administering to the subject a composition comprising a compound of the formula I: 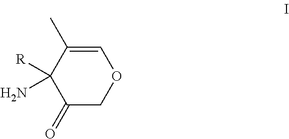
wherein R is C₂H₅ or C₂H₃,
or a pharmaceutically acceptable salt thereof.

9. The method according to claim 8, wherein the composition comprises an amount of 0.3 mg or more of compound of the formula I relative to 1 ml of the composition.

10. The method according to claim 8, wherein the composition is administered at single-dose level of 50 ml to 500 ml, and one time to twelve times a day.

11. The method according to claim 8, wherein the composition is administered by parenteral, topical, oral, rectal or nasal route.

12. The method according to claim 8, wherein the composition is formulated in shampoo, lotion, cream, gel, conditioner or essence.

13. The method according to claim 8, wherein the composition contains hydrophilic or lipophilic gelling agents, hydrophilic or lipophilic additives, preservatives, antioxidants, solvents, fragrances, fillers, screening agents, odor absorbers or coloring materials.

14. The method according to claim 8, wherein the composition is applied to the scalp.
